(19) United States
(12) Patent Application Publication
(10) Pub. No.: US 2017/0265799 A1
(43) Pub. Date: Sep. 21, 2017

(54) FETAL MOVEMENT MEASURING DEVICE

(71) Applicant: SOUTHERN TAIWAN UNIVERSITY OF SCIENCE AND TECHNOLOGY, Tainan City (TW)

(72) Inventors: YI-CHUN DU, TAINAN CITY (TW); JHENG-BANG SHIH, TAINAN CITY (TW); BEE-YEN LIM, TAINAN CITY (TW); WEI-SIANG CIOU, TAINAN CITY (TW); CHUNG-YI CHIU, TAINAN CITY (TW)

(21) Appl. No.: 15/460,453

(22) Filed: Mar. 16, 2017

(30) Foreign Application Priority Data
Mar. 17, 2016 (TW) 105108296

(51) Int. Cl. A61B 5/00; A61B 5/11

(52) U.S. Cl. CPC A61B 5/4362; A61B 5/6823; A61B 5/7271; A61B 5/11; A61B 5/0011; A61B 2503/02; A61B 2562/0219

(57) ABSTRACT

A fetal movement measuring device includes a wearable article to be worn on a pregnant woman's abdomen, plural measurement units, and a mobile device pre-installed with a fetal movement algorithm. The measurement units are provided separately on the outer surface of the wearable article and each include a fetal movement sensor for sensing a dynamic physiological signal of the abdomen and a power supply element for supplying necessary electricity to the fetal movement sensor. The dynamic physiological signals sensed by the fetal movement sensors are received by the mobile device, processed with the fetal movement algorithm, and rid of synchronous signal components. Then, the fetal movement algorithm performs calculation on the remaining signal components to generate fetal movement information, which includes a fetal movement location and a fetal movement magnitude. Thus, each fetal movement is measured in a non-contact manner, and its location, obtained through asynchronous multipoint measurement.

### BACKGROUND OF THE INVENTION

### 1. Technical Field

The present invention relates to a measuring device and more particularly to one configured for measuring fetal movement.

### 2. Description of Related Art

Fetal movement, uterine contraction, and fetal heart rate are three major physiological parameters by which to know a fetus's condition during its mother's pregnancy. Fetal movement refers to a fetus's movement in the uterus, uterine contraction refers particularly to the pressure generated by such contraction, and fetal heart rate refers to the speed of the fetus's heartbeats. Of the three parameters, fetal movement provides the earliest and most readily detectable signal and hence constitutes the most clinically recommended method for a pregnant woman to monitor her unborn baby's health by herself. This explains why pregnancy instruction manuals typically include a fetal movement record and encourage an expectant mother to measure fetal movement regularly to ensure the fetus's safety. However, measuring fetal movement by oneself is very time-consuming. During the eighth week of pregnancy, there is one fetal movement at least every 13 minutes. In the 20^{th} week, the average number of fetal movements per 12 hours is about 200, and the number increases to about 575 in the 32^{nd} week. For a pregnant woman, therefore, counting the number of fetal movements is by no means easy. Not only is the identification of fetal movement highly subjective, but also the prolonged counting process is difficult to carry out on a daily basis.

As a solution, Taiwan Patent No. 1267369, entitled “METHOD FOR AUTOMATIC FEEDBACK OF A PREGNANT WOMAN'S AND A FETUS'S PHYSIOLOGICAL STATE”, discloses using several different monitoring approaches and setting the monitoring cycle and times automatically. If the user responds well to the preset monitoring scheme, the default monitoring times remain. If the monitoring results prove unsatisfactory, the monitoring cycle or times will be modified, and the pregnant user, notified. By applying different monitoring approaches alternately, analyzing the results corresponding to different monitoring times and monitoring approaches as a whole, and repeating the monitoring process automatically based on automatic feedback, more detailed monitoring results can be obtained, allowing the pregnant user to be fully aware of the fetus's condition as well as her own and seek proper medical assistance when necessary. This patented method, however, is not designed for multipoint measurement and requires electrode pads to be attached to the user's skin in order to detect myoelectric signals, wherein the pads attached to the skin may cause discomfort to the user and are subject to interference. Besides, the '369 patent does not disclose how to determine, exclude, or prevent misjudgment attributable to, non-fetal movement that results from the user's own body movement, nor can the patented method locate each fetal movement; thus, the results and accuracy of fetal movement measurement leave something to be desired.

Taiwan Patent No. 1392480, entitled “APPARATUS AND METHOD FOR MATERNAL-FETAL SURVEILLANCE”, discloses a uterine contraction and fetal movement monitoring apparatus for monitoring a woman user's and a fetus's state. The monitoring apparatus includes a set of sensors, a signal pre-processor, a first signal post-processor, a first analyzing unit, a second signal post-processor, a second analyzing unit, and a third analyzing unit. The set of sensors are attached to the abdomen of a maternal body and provide at least three measuring leads. The signal pre-processor receives multiple sensing signals from the set of sensors, reduces noise in the signals, amplifies the characterizing signal portions, and outputs a set of characterizing signals. The first signal post-processor receives the set of characterizing signals from the signal pre-processor and filters out noise to obtain information related to the maternal body and the fetus, including electrocardiogram signals and uterine myoelectric signals of the maternal body and electrocardiogram signals of the fetus. The first analyzing unit is configured for calculating the fetus's sympathetic nerve activity level signal according to the information obtained by the first signal post-processor. The second signal post-processor receives the set of characterizing signals from the signal pre-processor and derives therefrom multiple fetal electrocardiogram complex waveforms and multiple uterine contraction signal waveforms that correspond to the leads respectively. The second analyzing unit analyzes the fetal electrocardiogram complex waveforms to obtain the fetal electrocardiogram complex waveform corresponding to each lead and a maternal electrocardiogram complex waveform, thereby determining whether there is a change in fetal position. The second analyzing unit also derives a uterine contraction state signal from the uterine contraction signals. The third analyzing unit determines the occurrence or absence of fetal movement by applying a fetal movement identification method, taking into account the uterine contraction state signal, the energy change signals, and the fetus's sympathetic nerve activity level signal, wherein the sympathetic nerve activity level signal serves to increase the accuracy of fetal movement identification. As the apparatus and method of the '480 patent still rely on electrode pads (i.e., sensors) attached to a pregnant user's skin to detect myoelectric signals, the discomfort and potential interference associated with the use of such pads remain. Moreover, the '480 patent does not disclose how to determine, exclude, or prevent misjudgment attributable to, non-fetal movement that results from the user's own body movement.

### BRIEF SUMMARY OF THE INVENTION

It is an objective of the present invention to provide a fetal movement measuring device that measures fetal movement by a non-contact method and that uses asynchronous multipoint measurement to locate each fetal movement.

The fetal movement measuring device of the present invention includes a wearable article, a plurality of measurement units, and a mobile device. The wearable article is configured to be worn on a pregnant woman's abdomen. The measurement units are provided separately on the outer surface of the wearable article. Each measurement unit includes a fetal movement sensor for sensing a dynamic physiological signal of the abdomen and a power supply element electrically connected to the fetal movement sensor to supply necessary electricity thereto. The mobile device is configured for receiving information from the fetal movement sensors and is pre-installed with a fetal movement algorithm. The mobile device receives the dynamic physiological signals sensed respectively by the fetal movement sensors and performs synchronous-signal analysis and determination through the fetal movement algorithm. When it is determined that the dynamic physiological signals have synchronous signal components, the mobile device removes the synchronous signal components, and the fetal movement algorithm performs calculation on the remaining signal components to generate fetal movement information, which includes a fetal movement location and a fetal movement magnitude.

The present invention is advantageous in that a pregnant woman only has to wear the wearable article and start the fetal movement measuring device, and fetal movement signals will be monitored continually, which is very convenient. Also, the measurement units are provided on the wearable article and therefore not in direct contact with the user's skin, which is a far cry from the conventional devices whose analysis is based on the measurement of physiological signals, such as those configured for analyzing myoelectric signals. Thus, the present invention provides enhanced comfort during use and is less susceptible to interference as compared with the prior art. Furthermore, as the present invention uses an asynchronous multipoint approach to calculate the location and time of each fetal movement, the accuracy of fetal movement measurement is higher than in the prior art, and the data obtained are of higher reference value in subsequent medical treatment than those obtained with the conventional devices.

### BRIEF DESCRIPTION OF THE SEVERAL VIEWS OF THE DRAWINGS

FIG. 1 schematically shows the fetal movement measuring device in the first embodiment of the present invention;

FIG. 2 is a block diagram showing how the components in the first embodiment are connected to one another;

FIG. 3 schematically shows a state of use of the first embodiment;

FIG. 4 schematically shows how a fetal movement wave propagates in the first embodiment;

FIG. 5a to FIG. 5d are oscillograms in support of FIG. 4, showing the dynamic physiological signals sensed respectively by a plurality of fetal movement sensors that are located at different positions, wherein the synchronous signal components have yet to be removed;

FIG. 6a to FIG. 6d are oscillograms similar to those in FIG. 5a to FIG. 5d except that the synchronous signal components have been removed;

FIG. 7 is a flowchart of the fetal movement algorithm in the first embodiment; and

FIG. 8 is a block diagram showing how the components in the second embodiment of the present invention are connected to one another.

### DETAILED DESCRIPTION OF THE INVENTION

The foregoing and others features and effects of the present invention will be detailed below with reference to some illustrative embodiments in conjunction with the accompanying drawings.

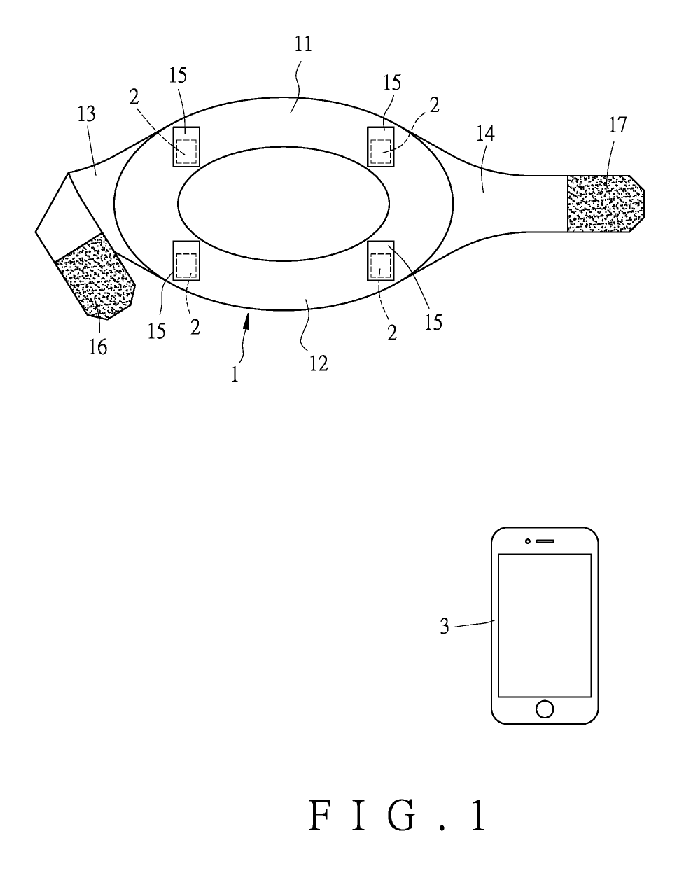

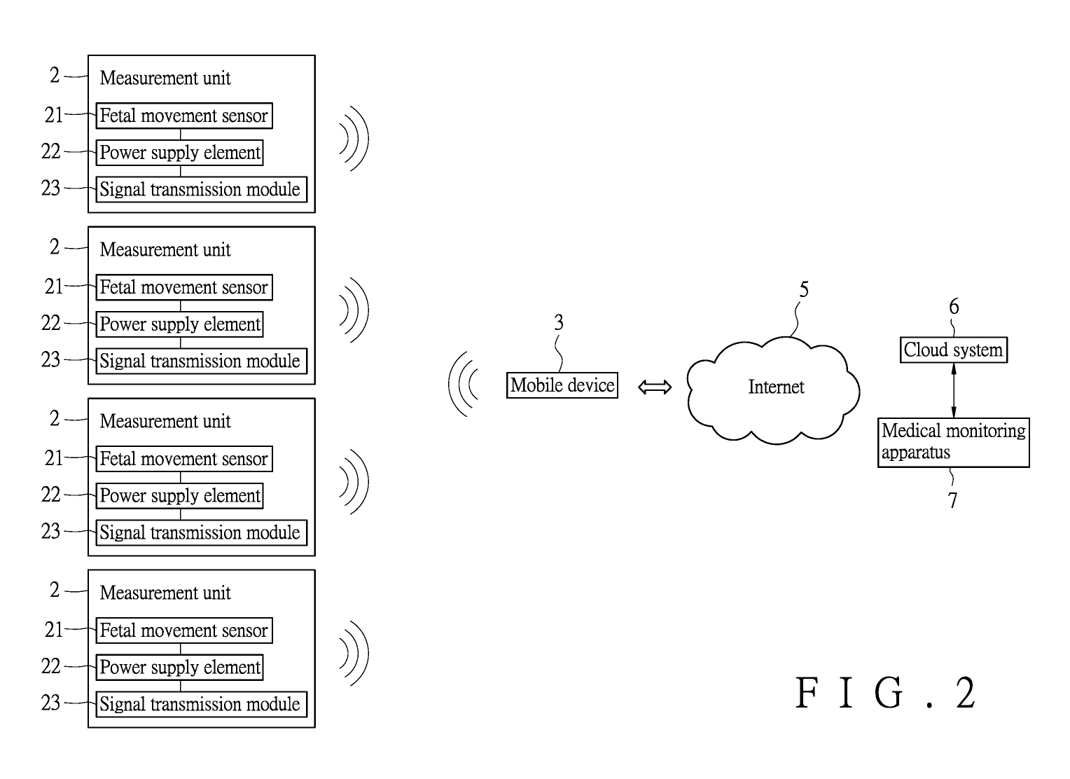

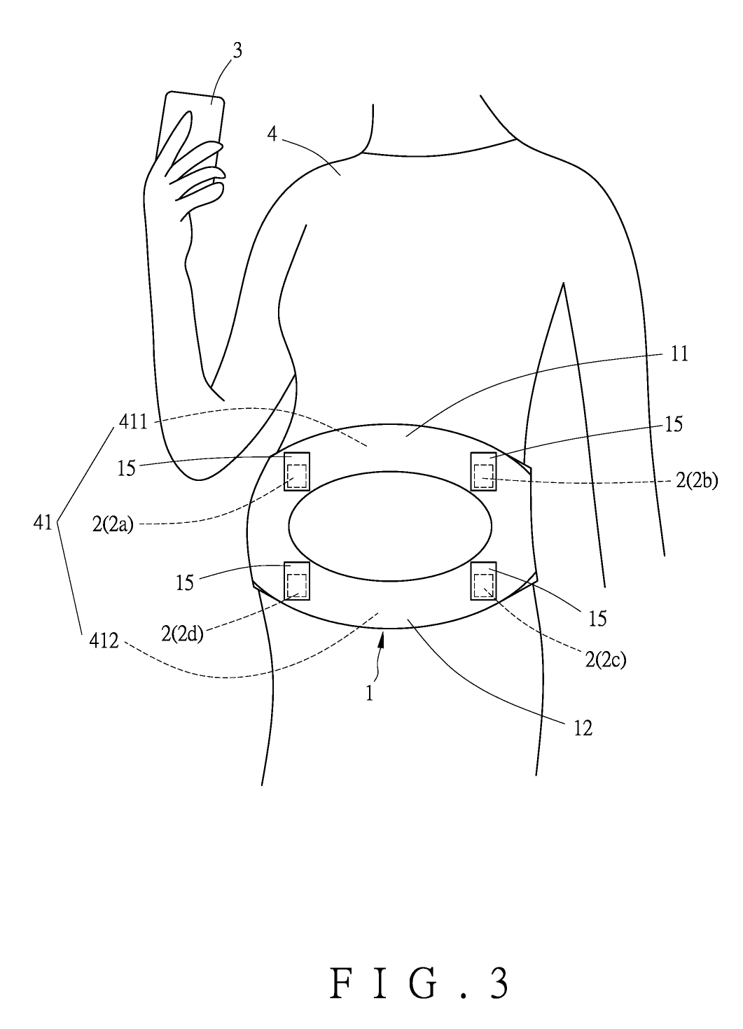

Referring to FIG. 1 and FIG. 2, the fetal movement measuring device in the first embodiment of the present invention includes a wearable article 1, a plurality of measurement units 2, and a mobile device 3. The wearable article 1 is designed to be worn on a pregnant woman 4's abdomen 41 as shown in FIG. 3 and is implemented in this embodiment as a maternity belt by way of example. The wearable article 1 includes an upper supporting piece 11 for covering an upper part 411 of the pregnant woman 4's abdomen 41, a lower supporting piece 12 for covering a lower part 412 of the pregnant woman 4's abdomen 41, a left connecting piece 13 connected between one end of the upper supporting piece 11 and one end of the lower supporting piece 12, and a right connecting piece 14 connected between the other end of the upper supporting piece 11 and the other end of the lower supporting piece 12. Each of the upper supporting piece 11 and the lower supporting piece 12 is provided with a plurality of pockets 15 on the outer surface. The pockets 15 correspond in number to the measurement units 2. In this embodiment, there are four pockets 15 and four measurement units 2 by way of example only; the present invention imposes no limitation on their quantities. Each pocket 15 is configured for receiving one of the measurement units 2. The left connecting piece 13 and the right connecting piece 14 are respectively provided with a first positioning member 16 and a corresponding second positioning member 17 where the two connecting pieces are to be connected with each other. Once the wearable article 1 covers the pregnant woman 4's abdomen 41, the first positioning member 16 on the left connecting piece 13 and the second positioning member 17 on the right connecting piece 14 can attach to each other to secure the wearable article 1 in place. It should be pointed out that the wearable article 1 is not limited to the maternity belt disclosed herein. For example, the upper supporting piece 11 and the lower supporting piece 12 may be integrated as a single piece that can cover the pregnant woman 4's entire abdomen 41 to enable fetal movement measurement. The wearable article 1 may also be formed as a common belt, provided that it can be worn on the pregnant woman 4's abdomen 41 and carry the measurement units 2.

In this embodiment, the measurement units 2 are respectively received in the pockets 15 on the outer surface of the wearable article 1, and yet assembly of the measurement units 2 and the wearable article 1 is not limited to the pocket-based design disclosed herein. For instance, clips or attaching elements may be used instead, as long as the elements or structures used can couple the measurement units 2 to the outer surface of the wearable article 1 in a detachable manner.

Each measurement unit 2 includes a fetal movement sensor 21 for sensing a dynamic physiological signal of the abdomen 41, a power supply element 22 electrically connected to the fetal movement sensor 21 to supply necessary electricity thereto, and a signal transmission module 23 electrically connected to both the fetal movement sensor 21 and the power supply element 22. The fetal movement sensors 21 may be inertia-based or pressure-based and in this embodiment are inertial measurement units (IMUs) by way of example, wherein each IMU includes a 3-axis accelerometer and a 3-axis gyroscope. The power supply elements 22 are batteries. Each fetal movement sensor 21 is configured for transmitting information to the mobile device 3 via the corresponding signal transmission module 23 by a wireless communication method.

In this embodiment, the mobile device 3 is implemented as a smartphone by way of example but is not limited thereto. For example, the mobile device 3 may alternatively be a tablet computer, a personal digital assistant, a smart watch, or the like. The mobile device 3 is pre-installed with a fetal movement algorithm for analyzing, making judgements about, and computing with the dynamic physiological signals of the pregnant women 4's abdomen 41 received respectively by the fetal movement sensors 21, in order to obtain accurate fetal movement information.

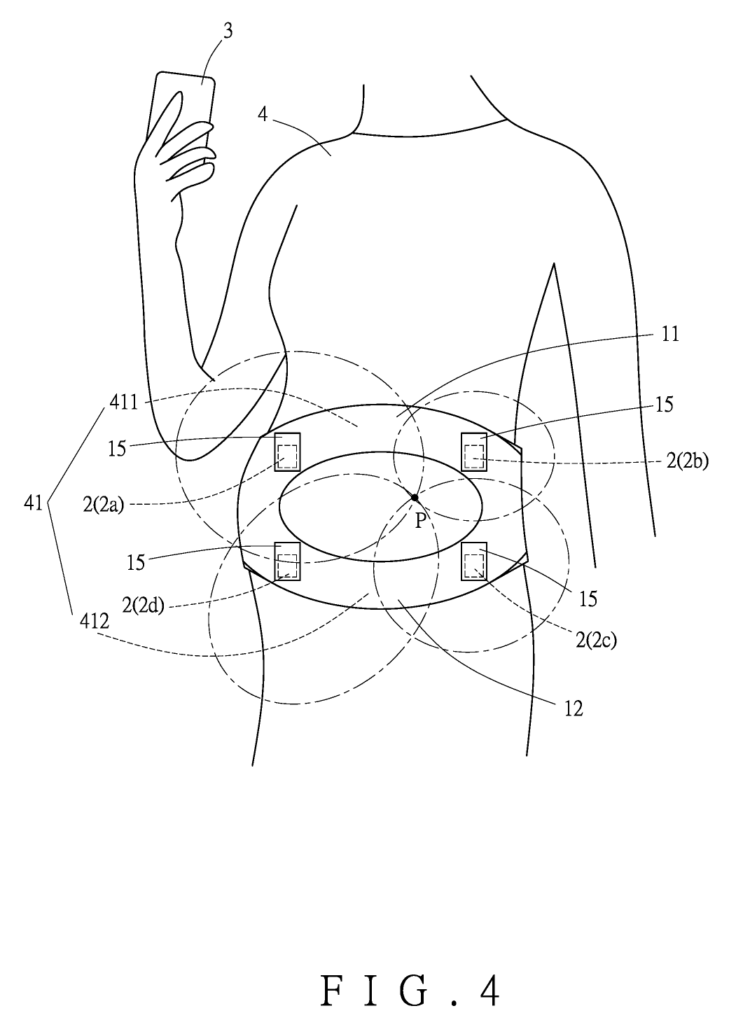

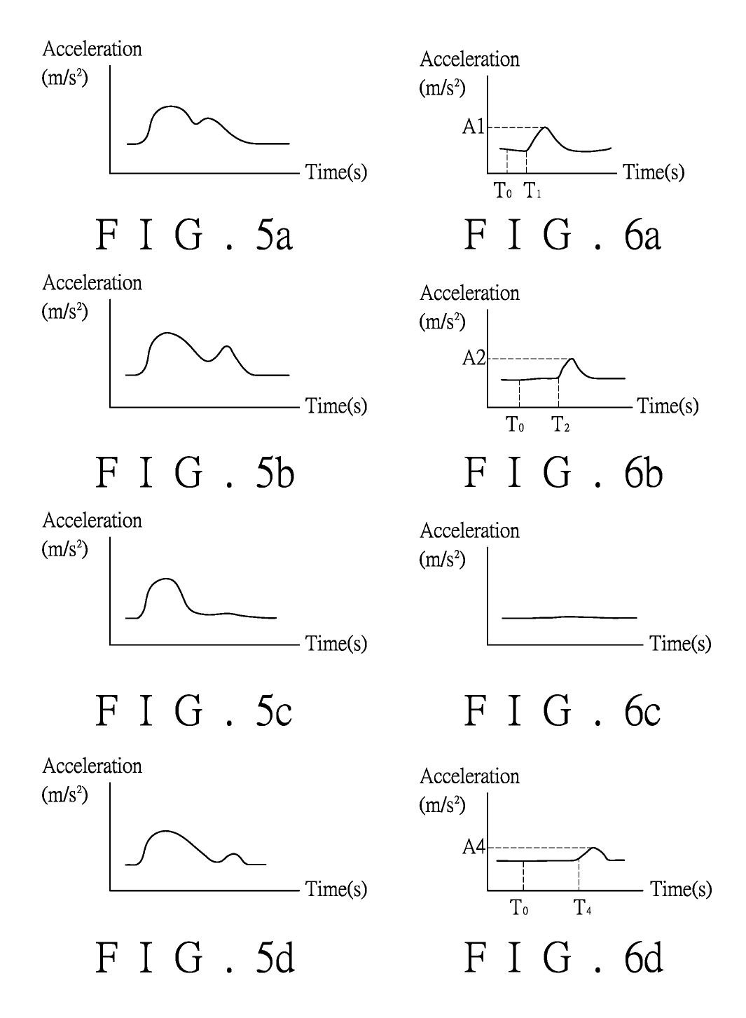

To use the fetal movement measuring device, referring to FIG. 1, FIG. 3, and FIG. 4, the pregnant woman 4 begins by covering her abdomen 41 with the wearable article 1. Then, the first positioning member 16 on the left connecting piece 13 and the second positioning member 17 on the right connecting piece 14 are attached to each other to complete the wearing process. The fetal movement measuring device can now be started so that the fetal movement sensors 21 of the measurement units 2 are calibrated and zeroed. After that, the measurement units 2, which are provided at different locations, will start sensing the dynamic physiological signals of different parts of the pregnant woman 4's abdomen 41 respectively. As shown in FIG. 4, when a fetal movement takes place at a point P, a fetal movement wave is generated and propagates through the amniotic fluid in the uterus. Since the distance from the fetal movement sensor 21 of each measurement unit 2 to the location the fetal movement (i.e., the point P) is different, the time at which the fetal movement wave is received and the magnitude of the fetal movement wave received vary from one sensor 21 to another. To facilitate description, the measurement units 2 are further numbered as 2a, 2b, 2c, and 2d respectively. When a fetal movement occurs at the point P, the measurement unit 2b is the closest to the location of the fetal movement, followed sequentially by the measurement unit 2c, the measurement unit 2a, and the measurement unit 2d. The dynamic physiological signals sensed respectively by the measurement units 2a, 2b, 2c, and 2d are plotted in FIG. 5a to FIG. 5d, with the vertical axis of each oscillogram representing acceleration in the unit of m/s² and the horizontal axis representing time in the unit of second (s).

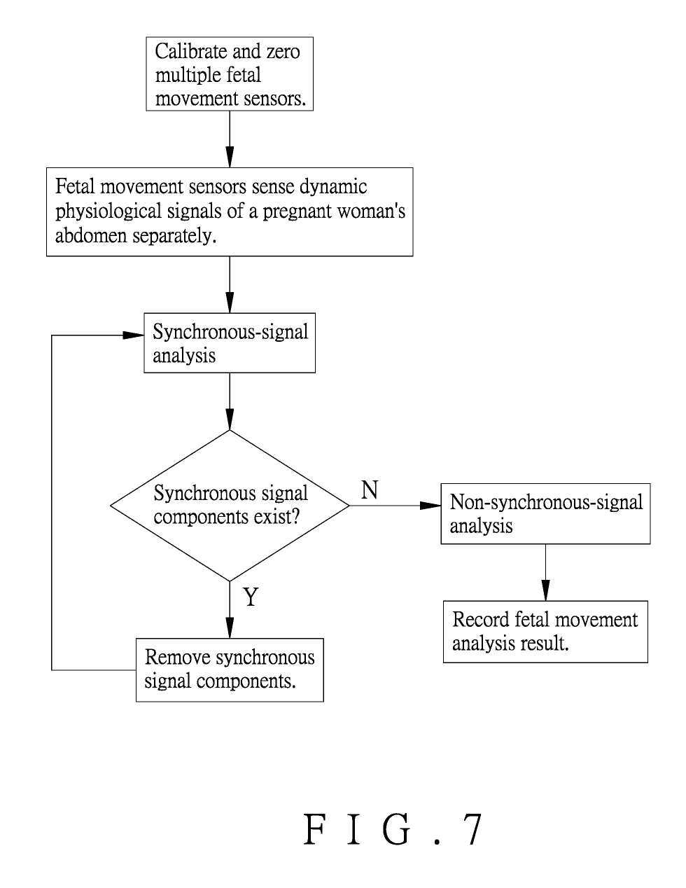

Each fetal movement sensor 21 transmits the dynamic physiological signal sensed to the mobile device 3 via the corresponding signal transmission module 23, and the mobile device 3 performs synchronous-signal analysis and determination on the dynamic physiological signals by executing the fetal movement algorithm. Referring to FIG. 7, when it is determined that the dynamic physiological signals have synchronous signal components, an adaptive filter is used to remove the synchronous signal components, and the remaining signal components are subjected to fetal movement analysis and calculation by the fetal movement algorithm to generate fetal movement information. It should be pointed out that synchronous-signal analysis should be performed again after the detected synchronous signal components are filtered out. This is because synchronous signal components cannot be completely filtered out in a single operation and require a second analysis, if not more. Here, fetal movement analysis is based on the difference in amplitude and propagation time of a vibration wave, as shown by equation (1), from which the fetal movement information is derived:

$$\frac{{T}_{u} - {T}_{0}}{V} = \sqrt{{( {X}_{0} - {X}_{n} )}^{2} + {( {Y}_{0} - {Y}_{n} )}^{2} + {( {Z}_{0} - {Z}_{n} )}^{2}} \qquad ( 1 )$$

where V is the propagation velocity of the vibration wave; T₀ is the time at which the fetal movement occurs; Tₙ is the time at which each fetal movement sensor 21 receives the vibration wave; n is an integer; X₀, Y₀, and Z₀ are coordinates of the location of the fetal movement; and Xₙ, Yₙ, and Zₙ are coordinates of the location of each fetal movement sensor 21. In this embodiment, four fetal movement sensors 21 are used, so n=1, 2, 3, or 4 is substituted separately into equation (1) to obtain the following equations (2) to (5):

$$\begin{matrix}\frac{{T}_{1} - {T}_{0}}{V} = \sqrt{{( {X}_{0} - {X}_{1} )}^{2} + {( {Y}_{0} - {Y}_{1} )}^{2} + {( {Z}_{0} - {Z}_{1} )}^{2}} & ( 2 ) \\ \frac{{T}_{2} - {T}_{0}}{V} = \sqrt{{( {X}_{0} - {X}_{2} )}^{2} + {( {Y}_{0} - {Y}_{2} )}^{2} + {( {Z}_{0} - {Z}_{2} )}^{2}} & ( 3 ) \\ \frac{{T}_{3} - {T}_{0}}{V} = \sqrt{{( {X}_{0} - {X}_{3} )}^{2} + {( {Y}_{0} - {Y}_{3} )}^{2} + {( {Z}_{0} - {Z}_{3} )}^{2}} & ( 4 ) \\ \frac{{T}_{4} - {T}_{0}}{V} = \sqrt{{( {X}_{0} - {X}_{4} )}^{2} + {( {Y}_{0} - {Y}_{4} )}^{2} + {( {Z}_{0} - {Z}_{4} )}^{2}} . & ( 5 )\end{matrix}$$

As T₀ and V₀ are fixed values, let T₀=0 and V=1, and equation (1) can be simplified as equation (6). By substituting n=1, 2, 3, or 4 separately into equation (6), the following equations (7) to (10) are obtained:

Tₙ²=(X₀−Xₙ)²+(Y₀−Yₙ)²+(Z₀−Zₙ)² (6)

T₁²=(X₀−X₁)²+(Y₀−Y₁)²+(Z₀−Z₁)² (7)

T₂²=(X₀−X₃)²+(Y₀−Y₂)²+(Z₀−Z₃)² (8)

T₃²=(X₀−X₃)²+(Y₀−Y₃)²+(Z₀−Z₃)² (9)

T₄²=(X₀−X₄)²+(Y₀−Y₄)²+(Z₀−Z₄)² (10)

With the X-, Y-, and Z-axis coordinates of the location of each fetal movement sensor 21 being aliquots, equation (6) can be further simplified as the following geometric equations (11) to (13):

(X₀−Xₙ₋₍ₙ₋₁₎)² . . . (X₀−Xₙ₋₂)²(X₀−Xₙ₋₁)²(X₀−Xₙ)²=Tₙ₋₍ₙ₋₁₎²:Tₙ₋₁²:Tₙ₋₁²:Tₙ² (11)

(Y₀−Yₙ₋₍ₙ₋₁₎)² . . . (Y₀−Yₙ₋₂)²(Y₀−Yₙ₋₁)²(Y₀−Yₙ)²=Tₙ₋₍ₙ₋₁₎²:Tₙ₋₂²:Tₙ₋₁²:Tₙ² (12)

(Z₀−Zₙ₋₍ₙ₋₁₎)² . . . (Z₀−Zₙ₋₁)²(Z₀−Zₙ₋₁)²(Z₀−Zₙ)²=Tₙ₋₍ₙ₋₁₎²:Tₙ₋₂²:Tₙ₋₁²:Tₙ² (13)

By substituting n=1, 2, 3, or 4 separately into equations (11) to (13), the following equations (14) to (16) are obtained, from which the coordinates of the location (X₀, Y₀, Z₀) of the fetal movement can be determined:

(X₀−X₁)²(X₀−X₂)²(X₀−X₃)²(X₀−X₄)²=T₁²:R₃²:T₃²:R₄² (14)

(Y₀−Y₁)²(Y₀−Y₂)²(Y₀−Y₃)²(Y₀−Y₄)²=R₂²:T₂¹:T₃²:T₄³ (15)

(Z₀−Z₁)²(Z₀−Z₂)²(Z₀−Z₃)²(Z₀−Z₄)²=T₁²:T₂²:T₃²:T₄² (16)

After determining the location of the fetal movement, the magnitude of the fetal movement can be calculated from the time at which each fetal movement sensor 21 receives the fetal movement wave and the largest amplitude detected by each fetal movement sensor 21, as shown by equation (17):

$$\frac{{A}_{0}}{\sqrt{{( {X}_{0} - {X}_{n} )}^{2} + {( {Y}_{0} - {Y}_{n} )}^{2} + {( {Z}_{0} - {Z}_{n} )}^{2}}} = {A}_{n} * k \qquad ( 17 )$$

where A₀ is the magnitude of the fetal movement, Aₙ is an amplitude, k is a correction coefficient, (X₀, Y₀, Z₀) indicates the location of the fetal movement, (Xₙ, Yₙ, Zₙ) indicates the location of each fetal movement sensor 21, and n is still an integer. In this embodiment, four fetal movement sensors 21 are used, so n=1, 2, 3, or 4.

Having obtained the fetal movement information, the mobile device 3 not only can display the information on its screen for view by the pregnant woman 4, but also can send the information through the Internet 5 to a cloud server 6 in order to be downloaded from the cloud server 6 and used by a medical monitoring apparatus 7.

The fetal movement measuring device is wearable and therefore readily adaptable to the pregnant woman 4's daily activities such as cooking, sleeping, shopping, and so on. Also, the measuring units 2 coupled to the wearable article 1 are compact, lightweight, and hence capable of measuring the number of fetal movements without affecting the pregnant woman 4's daily life. The pregnant woman 4 can do whatever she wants and never has to worry whether fetal movement has been measured. The mobile device 3 may be further provided with a detection module (not shown) and a prompt module (not shown) electrically connected to the detection module. The detection module is configured for detecting whether the pregnant woman 4 has put on the wearable article 1. If the detection module detects that the wearable article 1 is not worn by the pregnant woman 4, the prompt module will output a prompt sound, prompting or reminding the pregnant woman 4 to put on the wearable article 1 and wear it properly, lest the pregnant woman 4 forget to do so.

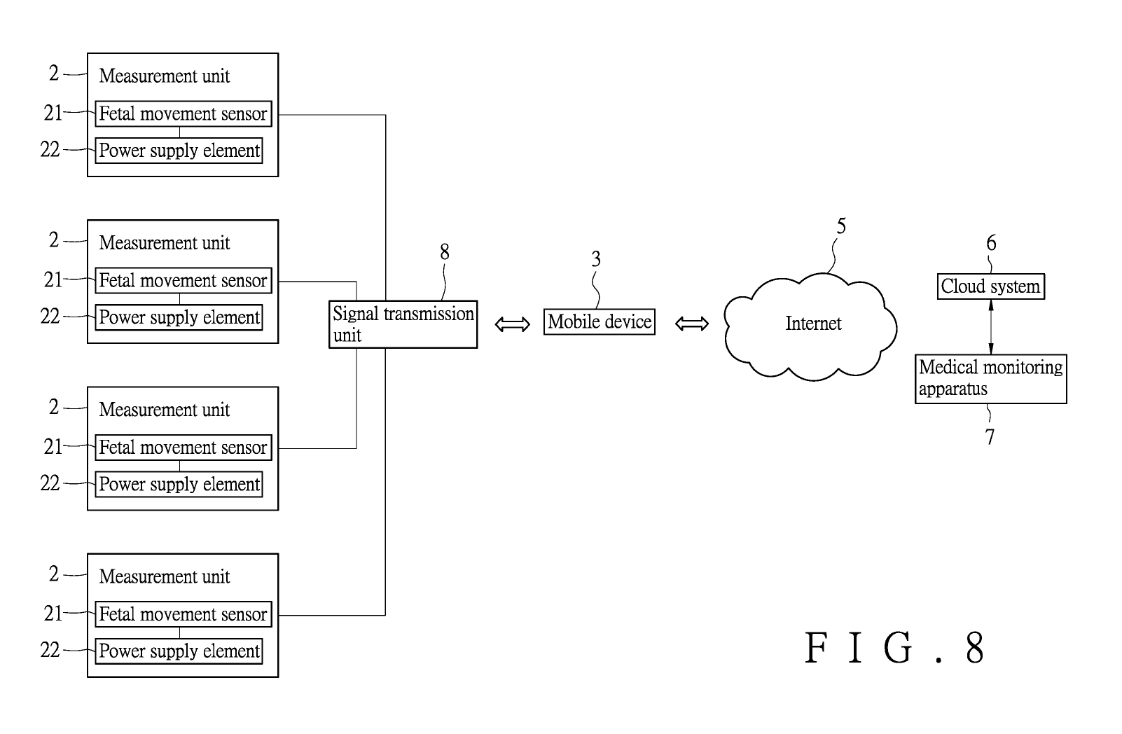

FIG. 8 shows the fetal movement measuring device in the second embodiment of the present invention. The second embodiment is generally the same as the first embodiment except that a signal transmission unit 8 electrically connected to the fetal movement sensors 21 is additionally provided, and that each measurement unit 2 only includes a fetal movement sensor 21 and a power supply element 22 electrically connected to the fetal movement sensor 21 to provide necessary electricity thereto. The signal transmission unit 8 can be put into one of the pockets 15 (see FIG. 1) along with the corresponding measurement unit 2 or be coupled to the outer surface of the wearable article 1 (see FIG. 1) independently. The second embodiment is equally capable of achieving the intended effects of the first embodiment.

In summary of the above, the fetal movement measuring device of the present invention is so designed that the pregnant woman 4 only has to wear the wearable article 1 and start the fetal movement measuring device, and fetal movement signals will be monitored continually without affecting the pregnant woman 4's daily life. As the measurement units 2 are provided separately on the outer surface of the wearable article 1 and measure fetal movement in a non-contact manner, i.e., without direct contact with the pregnant woman 4's skin, the present invention is more comfortable and less susceptible to interference than its prior art counterparts. Furthermore, the location and time of each fetal movement are obtained by asynchronous multipoint measurement to provide more accurate measurement results than conventionally achievable, and the data obtained are of higher reference value in subsequent medical treatment than those obtained in a traditional way. In addition, the mobile device 3 may be provided with a detection module and a prompt module so that when the detection module detects that the wearable article 1 is not worn by the pregnant woman 4, the prompt module will output a prompt sound to remind the pregnant woman 4 to put on the wearable article 1 and wear it properly. Thus, the fetal movement measuring device features great convenience of use on the whole.

The foregoing description of the embodiments should be able to enable a full understanding of the operation, use, and effects of the present invention. Those embodiments, however, are only some preferred ones of the invention and are not intended to be restrictive of the scope of the invention. All simple equivalent changes and modifications based on the contents of this specification and the appended claims should fall within the scope of the present invention.

What is claimed is:

1. A fetal movement measuring device, comprising:
a wearable article to be worn on a pregnant woman's abdomen;
a plurality of measurement units provided separately on an outer surface of the wearable article, wherein each said measurement unit includes a fetal movement sensor for sensing a dynamic physiological signal of the abdomen and a power supply element electrically connected to the fetal movement sensor to provide necessary electricity thereto; and
a mobile device configured for receiving information from the fetal movement sensors and pre-installed with a fetal movement algorithm, wherein upon receiving the dynamic physiological signals sensed respectively by the fetal movement sensors, the mobile device performs synchronous-signal analysis and determination through the fetal movement algorithm; and when it is determined that the dynamic physiological signals have synchronous signal components, the mobile device removes the synchronous signal components in order for the fetal movement algorithm to perform calculation on remaining signal components and thereby generate fetal movement information, the fetal movement information including a fetal movement location and a fetal movement magnitude.

2. The fetal movement measuring device of claim 1, wherein the mobile device obtains the fetal movement information by calculation according to the following equation:
$$\frac{{T}_{n} - {T}_{0}}{V} = \sqrt{{( {X}_{o} - {X}_{n} )}^{2} + {( {Y}_{0} - {Y}_{n} )}^{2} + {( {Z}_{0} - {Z}_{n} )}^{2}}$$
where V is a propagation velocity of a vibration wave; T₀ is a time at which a fetal movement occurs; Tₙ is a time at which each said fetal movement sensor receives the vibration wave and a corresponding said dynamic physiological signal caused thereby; n is an integer; X₀, Y₀, and Z₀ are coordinates of the fetal movement location; and Xₙ, Yₙ, and Zₙ are coordinates of a location of each said fetal movement sensor.

3. The fetal movement measuring device of claim 2, wherein by setting T₀=0 and V=1, a simplified version of the equation is obtained as follows:
Tₙ²=(X₀−Xₙ)²+(Y₀−Yₙ)²+(Z₀−Zₙ)².

4. The fetal movement measuring device of claim 3, wherein the coordinates of the location of each said fetal movement sensor are aliquots along an X axis, a Y axis, and a Z axis respectively such that the coordinates X₀, Y₀, and Z₀ of the fetal movement location can be determined with the following equations respectively:
(X₀−Xₙ₋₍ₙ₋₁₎)² . . . (X₀−Xₙ₋₂)²(X₀−Xₙ₋₁)²(X₀−Xₙ)²=Tₙ₋₍ₙ₋₁₎²:Tₙ₋₂²:Tₙ₋₁²:Tₙ²
(Y₀−Yₙ₋₍ₙ₋₁₎)² . . . (Y₀−Yₙ₋₂)²(Y₀−Yₙ₋₁)²(Y₀−Yₙ)²=Tₙ₋₍ₙ₋₁₎²:Tₙ₋₂²:Tₙ₋₁²:Tₙ²
(Z₀−Zₙ₋₍ₙ₋₁₎)² . . . (Z₀−Zₙ₋₂)²(Z₀−Zₙ₋₁)²(Z₀−Z₂)²=Tₙ₋₍ₙ₋₁₎²:Tₙ₋₁²:Tₙ₋₁²:Tₙ².

5. The fetal movement measuring device of claim 4, wherein after obtaining the fetal movement location, the fetal movement magnitude is determined with the following equation according to the time at which each said fetal movement sensor receives the vibration wave:
$$\frac{{A}_{0}}{\sqrt{{( {X}_{0} - {X}_{n} )}^{2} + {( {Y}_{0} - {Y}_{n} )}^{2} + {( {Z}_{0} - {Z}_{n} )}^{2}}} = {A}_{n} * k$$
where A₀ is the fetal movement magnitude, Aₙ is an amplitude, and k is a correction coefficient.

6. The fetal movement measuring device of claim 5, wherein the fetal movement sensors are inertial measurement units (IMUs).

7. The fetal movement measuring device of claim 6, wherein each said measurement unit further includes a signal transmission module electrically connected to a corresponding said fetal movement sensor and a corresponding said power supply element, and each said fetal movement sensor is configured for transmitting information to the mobile device via a corresponding said signal transmission module by a wireless communication method.

8. The fetal movement measuring device of claim 6, further comprising a signal transmission unit electrically connected to the fetal movement sensors, and the dynamic physiological signal sensed by each said fetal movement sensor is transmitted to the mobile device via the signal transmission unit by a wireless communication method.

9. The fetal movement measuring device of claim 7, wherein the mobile device is configured for sending the fetal movement information obtained to a cloud server through the Internet, in order for a medical monitoring apparatus to download the fetal movement information from the cloud server and use the fetal movement information.

10. The fetal movement measuring device of claim 8, wherein the mobile device is configured for sending the fetal movement information obtained to a cloud server through the Internet, in order for a medical monitoring apparatus to download the fetal movement information from the cloud server and use the fetal movement information.

11. The fetal movement measuring device of claim 1, wherein the power supply elements are batteries.
